(19) United States
(12) Patent Application Publication
(10) Pub. No.: US 2010/0178328 A1
(43) Pub. Date: Jul. 15, 2010

(54) COMBINATION THERAPY FOR OVARIAN CANCER

(75) Inventors: Ronald A. Martell, San Francisco, CA (US); David A. Karlin, Los Altos, CA (US); Hazel B. Breitz, Seattle, WA (US); Paul L. Weiden, Seattle, WA (US)

Correspondence Address: SCHWEGMAN, LUNDBERG & WOESSNER, P.A., P.O. BOX 2938, MINNEAPOLIS, MN, 55402, US

(73) Assignee: Poniard Pharmaceuticals, Inc., Seattle, WA (US)

(21) Appl. No.: 12/635,534

(22) Filed: Dec. 10, 2009

Related U.S. Application Data
(63) Continuation-in-part of application No. PCT/US08/08076, filed on Jun. 27, 2008.
(60) Provisional application No. 60/946,639, filed on Jun. 27, 2007.
(60) Provisional application No. 61/027,388, filed on Feb. 8, 2008.
(60) Provisional application No. 61/055,071, filed on May 21, 2008.

(51) Int. Cl. A61K 9/127; A61P 35/00; A61K 31/704; A61P 17/00

(52) U.S. Cl. 424/450; 514/34

(57) ABSTRACT

The present invention provides a method to treat ovarian cancer by the administration of effective amounts of picoplatin and pegylated liposomal doxorubicin.

### CROSS REFERENCE TO RELATED APPLICATIONS

This application is a continuation-in-part of PCT application PCT/US08/008076, filed Jun. 27, 2008, which in turn claims priority from U.S. Provisional Application Ser. Nos. 60/946,639 filed Jun. 27, 2007, 61/027,388 filed Feb. 8, 2008, and 61/055,071 filed May 21, 2008, all of which are incorporated by reference in their entireties herein.

### BACKGROUND

Picoplatin is a new-generation organoplatinum drug that has promise for treatment of various types of malignancies, including those that have developed resistance to earlier organoplatinum drugs such as cisplatin and carboplatin. Picoplatin has shown promise in the treatment of various kinds of cancer or tumor, including small cell lung cancer, colorectal cancer, and hormone-refractory prostate cancer.

Structurally, picoplatin is:

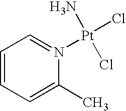

and is named cis-amminedichloro(2-methylpyridine)platinum(II), or alternatively [SP-4-3]-ammine(dichloro)(2-methylpyridine)platinum(II). The compound is a square planar complex of divalent platinum that is tetracoordinate and has three different ligand types. Two ligands are anionic, and two are neutral; therefore as the platinum in picoplatin carries a +2 charge, picoplatin is itself a neutral compound and no counterions need be present. The name “picoplatin,” referring to the presence of α-picoline (2-methylpyridine) in the molecule, is the United States Adopted Name (USAN), the British Approved Name (BAN), and the International Nonproprietary Name (INN) for this material. Picoplatin is also referred to in the literature as NX473, and is disclosed in U.S. Pat. Nos. 5,665,771, 6,518,428, and PCT/GB01/02060.

Studies in platinum-resistant ovarian and small cell lung cancer cell lines demonstrated the ability of picoplatin to overcome all three mechanisms of resistance. In cisplatin-resistant lung cancer cell lines, picoplatin uptake was maintained. Picoplatin has been shown in vitro to be significantly less susceptible than cisplatin to inactivation by thiol-containing compounds, such as thiourea and pyrimidine. Picoplatin remained active in four oxaliplatin-resistant colon and lung cell lines. Thus, picoplatin may also have particular utility against oxaliplatin resistant tumors. Picoplatin can be effective both in the treatment of resistant tumors that have failed prior platinum therapy as well as in the treatment of tumors not previously exposed to a platinum analogue.

Plasma pharmacokinetics following intravenous (IV) administration of picoplatin to the mouse, rat and dog showed a bi-exponential decay in plasma with rapid distribution followed by slow elimination (t_{1/2} of 44, 40 and 60 hours respectively). Platinum was rapidly and widely distributed into tissues of the mouse (with the exception of the brain).

Tetracoordinate square planar platinum (II) complexes are well known to be subject to oxidation to octahedral Pt(IV) complexes, such as with molecular chlorine. Also, it is well known that square planar platinum (II) complexes are subject to axial attack in ligand displacement reactions by various nucleophiles such as halides, amines, thio compounds, and under some conditions, water. Therefore, while picoplatin is relatively stable in pure form, in the absence of light, it can be subject to degradation under certain conditions, such as in the presence of nucleophilic molecular entities, particularly when in solution. It is known that picoplatin can decompose through formation of an aquo complex resulting from displacement of a chloride ion by water. See Advanced Inorganic Chemistry, F. Albert Cotton and Geoffrey Wilkinson, Second Revised Edition (1966) and later editions, Interscience Publishers. When administered to patients, picoplatin is believed to undergo metabolic transformation to some extent to two distinct aquo forms resulting from displacement of either of the chloride ligands. These cationic species (cationic as a result of displacement of a chloride anion by neutral water) are reactive, and interact with cellular DNA to bring about cross-linking and eventual cell death. Picoplatin is also known to be unstable in the presence of certain transition metal oxides, such as titanium dioxide and iron oxide.

Picoplatin's low stability in water, instability to light and certain metal salts, toxicity and teratogenicity pose obstacles to the preparation of effective liquid dosage forms. Therefore there is a continuing need for effective and stable dosage forms of picoplatin for both parenteral and oral administration.

### SUMMARY OF THE INVENTION

The present invention provides a method of treatment of platinum refractory, progressive, or recurrent ovarian cancer, comprising, administering to a human patient afflicted with ovarian cancer, substantially concurrently; picoplatin and pegylated liposomal doxorubicin hydrochloride (“LDR” or “liposomal doxorubicin”), preferably Doxil®, wherein the picoplatin is administered at least once at a dosage of at least about 60 mg/m² and the liposomal doxorubicin hydrochloride is administered at least once at a dosage of at least about 20 mg/m² of doxorubicin hydrochloride, up to the maximum tolerated dose of each agent in combination.

The invention also provides a method of inhibiting the growth of tumor cells in a human afflicted with ovarian cancer that comprises administering to such human an effective tumor cell growth inhibiting amount of picoplatin and an effective tumor cell growth inhibiting amount of liposomal doxorubicin hydrochloride, wherein the picoplatin and the liposomal doxorubicin hydrochloride are administered substantially concurrently.

The present invention further provides a kit comprising packaging containing, separately packaged, a sufficient number of unit dosage forms of picoplatin and a sufficient number of unit dosage forms of liposomal doxorubicin hydrochloride to provide for a course of treatment of for a human afflicted with ovarian cancer, along with instructional materials describing the dosing regimens disclosed herein.

Preferably, the administration of the picoplatin is prior to administration of the doxorubicin (e.g., sequential, including separately, and/or concurrently). Preferably the administration of the picoplatin and the liposomal doxorubicin is repeated for a plurality of treatments (e.g., about once every 3 to 6 about weeks for about 2 to about 10 treatments).

The present invention preferably carries out by the administration of stabilized liquid dosage forms of the anticancer drug picoplatin. The dosage forms of the invention can be adapted for parenteral administration or for oral administration.

Various embodiments of the invention provide a dosage form for picoplatin, wherein the picoplatin is stabilized against hydrolytic degradation. In various embodiments, chloride ion in a pharmaceutically acceptable form is present in a pH-adjusted, aqueous solution of picoplatin, the chloride ion being present in concentrations sufficient to reduce the hydrolytic degradation of the picoplatin. In various embodiments, the chloride ion is present at a concentration of at least about 9 mM. In various embodiments, the chloride ion can be provided by a pharmaceutically acceptable chloride salt, such as sodium chloride, potassium chloride, magnesium chloride, calcium chloride, or a combination thereof Or, the chloride ion can be provided by hydrochloric acid. The pH of the dosage form can be adjusted by titration with hydrochloric acid and sodium hydroxide.

Various embodiments of the invention provide a method for preparing a stabilized aqueous dosage form of picoplatin, that preferably is aseptic, or sterile. In various embodiments, the inventive methods comprise dissolving chloride ion as contained in a suitable salt or acid form in an aqueous solution of picoplatin, wherein the amount of chloride ion is effective to stabilize the picoplatin in aqueous solution, such as against hydrolytic degradation. The effective concentration of chloride ion can be no less than about 9 mM. The chloride concentration can range up to at least about 155 mM (isotonic) or higher. The effective chloride ion concentration can be achieved through the presence in the solution of at least about 0.05 wt % sodium chloride, ranging up to about 0.9% (isotonic), or even higher, provided the concentration used is not toxic. In various embodiments, aqueous solutions containing 2-5 wt % sodium chloride may be used, and diluted prior to use, or directly infused. The sodium chloride can be added to the solution in salt form, or can be prepared in situ by addition of a suitable amount of hydrochloric acid and titration with sodium hydroxide solution. Other sources of chloride ion can also be used.

Unexpectedly, it has been found that such solutions, when sealed and maintained under ambient conditions, will both maintain sterility indefinitely or, if not sterile, e.g., not aseptic initially, will gradually self-sterilize, eliminating all detectable microorganisms, e.g., bacteria, and will become aseptic without the need for added biocides or biocidal treatments, such as heat or irradiation.

Various embodiments of the invention provide a kit comprising a vial, infusion bag, or syringe, containing an inventive dosage form, or a dosage form prepared by an inventive method. The kit can further include instructional material and accessories useful for administering the dosage form.

Various embodiments of the invention provide methods of treatment of a cancer in a patient in need thereof, the methods comprising administration of an inventive stabilized aseptic dosage form of picoplatin, or a stabilized dosage form of picoplatin prepared by an inventive method, in an effective amount to the patient. The cancer-afflicted patient can be chemotherapy-naive, or can previously have received therapies (cancer therapy or radiation) that proved to be ineffective in controlling the patient's cancer. In various embodiments, the dosage form can be administered parenterally, such as by intravenous infusion, or can be administered orally. In various embodiments, the cancer can be refractory or progressive lung cancers (Small Cell Lung Cancer (SCLC) or Non Small Cell Lung Cancer (NSCLC)), breast cancer, colorectal cancer, head and neck cancer, renal cell cancer, gastric cancer, bladder cancer, liver cancer, mesothelioma, ovarian cancer, sarcoma such as leiomyosarcoma, thymic cancer, pancreatic cancer, peritoneal cancer, or prostate cancer.

In various embodiments, the stabilized picoplatin dosage form can be administered to the patient in combination with other anticancer agents in various regimens. In various embodiments, the stabilized picoplatin dosage form does not cause severe neuropathy as a side effect, or only causes low levels of neuropathy, i.e., grade 1 or 2 neuropathy only or infrequent neuropathy.

### DETAILED DESCRIPTION OF THE INVENTION

In various embodiments, the concentration of chloride ion, such as provided in the form of sodium chloride, in the stabilized dosage form is selected so as to provide a concentration of chloride ion in aqueous solution sufficient to reduce the degradation of the picoplatin through loss of chloride ion and conversion to aquo complexes. As shown below, it is believed that picoplatin undergoes a hydrolytic reaction in the presence of water, yielding degradation products, such as those designated “Aquo 1” and “Aquo 2” as shown below.

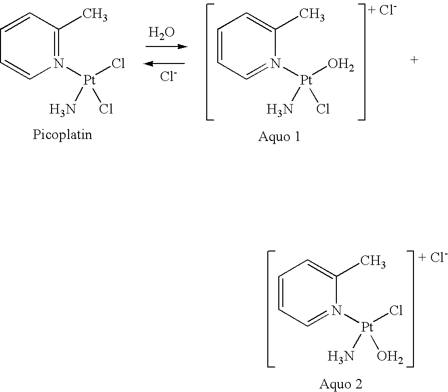

It is believed by the inventors herein that the presence of chloride ion serves to stabilize picoplatin in aqueous solution by driving the equilibrium to the left, such as by a mass action effect. In various embodiments, the chloride ion can be present in concentrations of at least 9 mM, corresponding to a sodium chloride concentration of about 0.05 wt % in the solution. The chloride ion can be present in concentrations ranging up to about 155 mM, or about 0.9 wt % of NaCl, an isotonic concentration, or alternatively, to concentrations of greater than about 155 mM, higher than an isotonic concentration, as long as the concentration used is not toxic to the patient. For example, about 1-5 wt-%, e.g., 2.5-3 wt-% sodium chloride can be present in some formulations.

In various embodiments, the inventive stabilized picoplatin solution can be prepared by dissolving an appropriate amount of picoplatin in water and providing an effective amount of chloride ion. In various embodiments, the solution pH can be adjusted, for example to about 5.5-6.0, such as with hydrochloric acid and sodium hydroxide. Picoplatin in any suitable physical form can be dissolved in water. For example, picoplatin can be added in the form of a micronized powder to the water solvent. The micronized powder can consist of amorphous picoplatin particles of less than about 10μ in average diameter, e.g., of about 2-5μ in diameter. These micronized picoplatin particles can be prepared by a variety of methods such as jet-milling, lyophilization, or microcrystallization. An aqueous picoplatin solution of about 0.5-1.1 mg/ml can result, which can be stabilized by addition of an effective amount of chloride ion, such as in the form of sodium chloride, or potassium chloride, or magnesium chloride, or any pharmaceutically acceptable form of chloride ion wherein the cationic counterion does not react significantly with picoplatin. The pH of the solution can be adjusted, for example to a pH of about 5.5-6.0, e.g., using hydrochloric acid and sodium hydroxide solutions.

Picoplatin is the cis-dichloro isomer of the molecular formula as depicted hereinabove. This isomeric form can be essentially free of the trans-isomer, e.g., the picoplatin can be at least 99.9% isomerically pure. The synthetic method used to prepare the cis-isomer can be selected to yield cis-isomer that is at least of this degree of purity. See U.S. Pat. No. 6,518,428. Alternatively, less isomerically pure picoplatin can be purified to remove any substantial amounts of the trans-isomer.

It has been unexpectedly found by the inventors herein that presence of chloride ion in an aqueous solution of picoplatin, such as relatively low concentrations of dissolved sodium chloride, which can be no less than about 0.05 wt %, can reduce the amount or rate of conversion of the picoplatin to the aquated, dechlorinated species in aqueous solution. The chloride ion, from whatever source, can be present in the solution at concentrations of no less than about 9 mM. In picoplatin solutions at pH 5.8 or less in the presence of chloride ion concentrations in this range, the amount or rate of conversion of picoplatin into the Aquo 1 and Aquo 2 forms is reduced relative to the amount or rate of conversion of the picoplatin in the absence of chloride ion. For example, in the inventive dosage form, Aquo 1 can be present at no more than about 2.5 wt % of the total dissolved picoplatin present, and Aquo 2 can be present at no more than about 2 wt % of the total dissolved picoplatin. These values correspond to concentration of the Aquo species in the aqueous solution of about 0.002 wt % and about 0.0015 wt % respectively for a 0.075 wt % solution of picoplatin. In other words, the two isomeric mono-dechlorinated complexes [(ammine)(chloro)(aquo)(2-picoline)]Pt(II) together amount to no more than about 4.5% wt % of the total dissolved picoplatin at pH 5.8, in the presence of no less than about 0.5 wt % NaCl, which is significantly lower than the amount of the mono-dechlorinated complexes that are formed in the absence of added chloride ion.

The inventors herein have found that the pH of the solution can be maintained at about 6 or less, for example at a pH of 5.0 to 6.0, or even less. In various embodiments, the picoplatin solution does not comprise an organic acid. For example, the solution can include HCl and NaOH to adjust the pH to the desired point and to provide chloride ions in the solution to achieve the stabilization effect. At this pH, the bioactivity of the solution is not adversely affected, and the solution is storage-stable. If lower pH values are used for storage of a picoplatin, e.g., pH 2-4, the pH can be raised closer to physiological pH prior to administration to a patient, for example by titration with inorganic bases such as sodium hydroxide.

The dosage form can comprise, in a container comprising a suitable closure means, an aseptic aqueous solution comprising (a) a preselected amount of dissolved picoplatin; (b) water; and (c) chloride ion, such as from the presence of NaCl, in an amount effective to stabilize the picoplatin. For example, picoplatin-compatible reagents can be used to adjust the pH, such as NaOH/HCl. The pH of the solution can be adjusted by titration of a solution incorporating HCl with a pharmaceutically acceptable inorganic base such as NaOH.

The inventive picoplatin dosage form can be used to treat cancers, such as solid tumors treatable by picoplatin, such as refractory or progressive lung cancers (Small Cell Lung Cancer (SCLC) or Non Small Cell Lung Cancer (NSCLC), breast cancer, colorectal cancer, head and neck cancer, renal cell cancer, gastric cancer, bladder cancer, liver cancer, mesothelioma, ovarian cancer, sarcoma such as leiomyosarcoma, thymic cancer, pancreatic cancer, peritoneal cancer, or prostate cancer. The dosage form can be administered parenterally, or can be administered orally. The dosage form can be used in combination with other anticancer agents. The dosage form can be used for adjuvant or first-line treatment of cancers (i.e., administered to a chemotherapy-naïve patient), or in second or third+-line treatment of cancers (i.e., when an initial course of chemotherapy with platinum or non-platinum agents has failed to induce remission in the cancer, for example when the cancer is refractory to initial chemotherapy or when the cancer is progressive following subsequent course or courses of chemotherapy). Picoplatin does not cause severe neuropathy, or infrequent neuropathy, or else only causes lower levels of neuropathy, as a side effect; no neuropathy of grade 3 or higher is caused by the picoplatin.

The composition of one such solution adapted for intravenous administration, to be held in the 200 mL container of an embodiment of the dosage form, is shown in the table below.

TABLE 1
Qualitative Composition of Picoplatin Intravenous Infusion

| Ingredient | Function |
|---|---|
| Picoplatin, 0.05 wt-% | Active Ingredient |
| Sodium Chloride USP, 0.9 wt-% | Stabilizer |
| Water for Injection USP, q.s. | Solvent |

Other suitable tonicity adjusters such as MgCl₂, CaCl₂, KCl, and the like, or non-ionic tonicity adjusters such as carbohydrates and sugar alcohols and the like, can be used in place of or in addition to sodium chloride. The sodium chloride is present in at least about 0.05 wt % (9 mM chloride ion; 0.05 wt % NaCl=8.5 mM NaCl: as calculated 0.05 gm/100 mL water−>0.5 gm/L; MW NaCl=58.5; 0.5/58.5=0.0085M=approx. 9 millimolar (mM)) to provide the picoplatin stabilization, but tonicity adjustments can be made using substances comprising or not comprising chloride ion to yield an isotonic solution adapted for IV administration. When sodium chloride is the sole tonicity adjuster, it can be present at about 0.9 wt % (i.e., about 154 mM) to provide an isotonic solution adapted for IV administration. Alternatively, the sodium chloride can be present in concentrations of greater than about 0.9%. For IV administration, the chloride concentration can be lower and the tonicity adjustment made with other compounds, such as non-ionic compounds, for example carbohydrates or sugar alcohols. For example, tonicity can be adjusted with sugar alcohols such as mannitol or sorbitol. For compositions adapted for oral administration, tonicity need not be adjusted, and provided that chloride ion is present in concentrations of at least about 9 mM (0.05 wt % NaCl) no other ingredients need be present.

The present invention also provides a solid composition prepared by lyophilizing the solution comprising picoplatin, a chloride ion source and a second stabilization agent such as a sugar alcohol, e.g., mannitol, sorbitol and the like. The composition is stable and can be reconstituted with water to yield an IV infusible solution, or a solution adapted for oral administration. A solution that is IV infusible can be isotonic. Lyophilizing or otherwise removing water from the inventive dosage form can provide a composition that is highly stable on storage but can readily be reconstituted to the desired concentration by re-addition of water.

Both the container and the water can be free of significant amounts of aluminum and/or transition metal salts and other compounds that can complex and/or otherwise degrade or reduce the activity of the picoplatin.

Suitable containers for the inventive dosage form include glass infusion vials, for example, nominal 150-225 mL vials, such as 200 mL vials, infusion bags formed of a compatible plastic such as ethylene-vinyl acetate copolymer, or polypropylene syringes adapted for intravenous administration of said solution. In another embodiment of the invention, the container is further enclosed or packaged in an opaque covering. Also, the glass or polymer of which the container is formed can be colored, e.g., amber colored, to provide further shielding from light exposure. Accordingly, various embodiments of the invention provide a kit comprising a vial, infusion bag, or syringe, such as are described above, containing an inventive dosage form, or a dosage form prepared by an inventive method. The kit can further include instructional material

The solution of the inventive dosage form is stable if stored or maintained at about 0.5-40° C. The solution may be stored at about 20-25° C. (about 68-77° F.), but may be stored at lower temperatures, e.g., at refrigerator temperatures of about 4-8° C., preferably under an inert atmosphere. Similarly, the lyophilized or otherwise dehydrated composition can be stored at these temperatures, and can also be stored at sub-zero (Celsius) temperatures to provide even greater stability over time.

The dosage form can be aseptic, and can be free of a preservative or biocide, such as a chlorite, chlorine dioxide, parabens or quarternary ammonium salt, that can react with the picoplatin and interfere with its bioactivity. Unexpectedly, the present dosage forms self-sterilize, in that they eliminate detectable microorganisms when maintained in the above described packaging, sealed and under ambient conditions.

In another embodiment of the invention, the present dosage form is enclosed in packaging with instruction materials, such as paper labeling, a tag, a compact disk, a DVD, a cassette tape and the like, regarding administration of the dosage form to treat SCLC. For example, the instruction materials can comprise labeling describing/directing a use of the dosage form that has been approved by a government agency responsible for the regulation of drugs.

The invention further provides a kit adapted for a single course of treatment comprising two or more, e.g., 2-3, containers as described above enclosed in packaging material, for example polystyrene foam packaging adapted to protect the bottles from impact, light, extremes of temperature, and so forth. The kit can further include accessories useful for administration of the container contents such as tubing, valves, needles for IV administration, etc. A kit can further include instructional materials, such as instructions directing the dose or frequency of administration. For example, a kit can comprise sufficient daily doses for a prolonged period, such as a week or a plurality of weeks, or can comprises multiple unit dosage forms for a single administration when the dose is to be repeated less frequently, such as a daily dose. The multiple unit dosage forms can be packaged separately, but in proximity, as in a blister pack. The kit can also include separately packaged, a plurality of unit dosage forms of the non-platinum containing anti-cancer agent, preferably oral unit dosage forms. The invention further provides a plurality of kits in a packaging adapted for shipping, for example, two courses of three containers each.

The method of treatment of the invention can further include orally or parenterally administering, preferably sequentially (before or after) or concurrently (including simultaneously or overlapping), at least one additional medicament and/or anti-cancer therapy, including radiation therapy, with a unit dosage form or a plurality of unit dosage forms comprising picoplatin, such as the unit dosage form(s) of the invention or prepared by the method of the invention. The additional medicament can be an anti-cancer medicament, preferably a non-Pt containing medicament, and may be administered orally or intravenously. Preferably, the administration is carried out so that effective amounts of picoplatin and the second (or third) medicament are present in vivo at the same time.

The kit can also contain one or more containers of solution of a second, platinum- or non-platinum anticancer drug and/or an adjunct agent, such as a potentiation agent (leucovorin), rescue agent (folate), anti-emetic (palenosetron), and the like. The first (picoplatin) and second container can be provided with fluid delivery means to permit the simultaneous administration to a cancer patient of solutions from both containers.

In various embodiments, the present invention provides a method for treating cancer comprising administering an inventive dosage form or a dosage form prepared by an inventive method to a patient afflicted by cancer, in an amount, at a frequency, and for a duration of treatment effective to provide a beneficial effect to the patient. For example, the dosage form can be administered orally, or the dosage form can administered intravenously to the patient. The patient can be chemotherapy-naïve or the patient can have previously received chemotherapy. The cancer can comprise a solid tumor, refractory or progressive lung cancers (Small Cell Lung Cancer (SCLC), Non Small Cell Lung Cancer (NSCLC)), colorectal cancer, breast cancer, head and neck cancer, renal cell cancer, gastric cancer, bladder cancer, liver cancer, mesothelioma, ovarian cancer, sarcoma such as leiomyosarcoma, thymic cancer, pancreatic cancer, or prostate cancer.

In various embodiments, a method for treating cancer comprising administering at least one liquid unit dosage form of picoplatin parenterally, by injection or infusion, to a human afflicted with cancer, to provide an effective therapeutic amount of picoplatin in one or more treatment cycles, is provided. The picoplatin can be administered in combination with (before, after or concurrently with) at least one platinum or non-platinum anti-cancer agent, which can be administered orally or parenterally.

In various embodiments, the stabilized dosage form of picoplatin can be administered orally. The picoplatin can be used to treat cancer in combination with (before, after or concurrently with) at least one platinum or non-platinum anticancer agent, which can be administered orally or parenterally. Additive effects between the picoplatin and the additional anticancer agent can be observed, wherein the therapeutic effect of each agent is summed to provide a proportional increase in effectiveness. Synergistic effects between the picoplatin and the additional anticancer agent can be observed, wherein the combined effectiveness of the treatment is greater than the summed effectiveness of the two agents.

In various embodiments of the invention, a method is provided for the treatment of cancer, such as lung cancer including small cell lung cancer (SCLC) and non-small cell lung cancer (NSCLC), kidney cancer, bladder cancer, renal cancer, stomach and other gastrointestinal (GI) cancers, mesothelioma, melanoma, peritoneal lymphoepithelioma, endometrial cancer, glioblastoma, pancreatic cancer, cervical cancer, testicular cancer, ovarian cancer, colorectal cancer, esophageal cancer, uterine cancer, endometrial cancer, prostate cancer, thymic cancer, breast cancer, head and neck cancer, liver cancer, sarcomas, including Kaposi's sarcoma, carcinoid tumors, other solid tumors, lymphomas (including non-Hodgkins lymphoma, NHL), leukemias, bone-associated cancers and other cancers disclosed in the patents and patent applications cited hereinbelow. For example, the present method can be used to treat small cell lung cancer (SCLC), hormone refractory prostate cancer (HRPC), colorectal cancer, or ovarian cancer, as a first-line treatment, or alternatively, to treat SCLC, hormone refractory prostate cancer (HRPC), colorectal cancer, or ovarian cancer, that is refractory to initial treatment or that is responsive to initial treatment but then progresses following cessation of the initial treatment. In various embodiments, the stabilized picoplatin dosage form can be administered as the only chemotherapeutic anti-cancer agent, in doses spaced at about three- to six-week intervals, wherein at least two doses are administered. Alternatively, as discussed below, additional chemotherapeutic agents and/or radiation therapy can be administered in conjunction with the picoplatin dosage form.

For example, an additional anti-cancer medicament can comprise, without limitation, a taxane (e.g., paclitaxel or docetaxel), a tyrosine kinase and/or a growth factor receptor inhibitor such as a VEGFR inhibitor (e.g., an antibody such as monoclonal antibodies bevacizumab (Avastin®), trastuzumab (Herceptin®), panitumumab (Vectibix®) or cetuximab (Erbitux®); a cephalotaxine analog (irinotecan), cediranib also known as AZD2171 (Recentin®), erlotinib (Terceva®) or sunitinib (Sutent®), an anti-metabolite (capecitabine, gemcitabine or 5-FU with or without leucovorin), a PK inhibitor (e.g., sorafenib tosylate, Nexavar®), dasatinib (Sprycel®), gefitnib (Iressa®) , imatinib (Gleevac®), lapatinib (Tykerb®), an anthracyclin (amrubicin, doxorubicin or liposomal doxorubicin), a Vinca alkaloid, or an alkylating agent, including melphalan and cyclophosphamide. Alternatively, the additional medicament is a non-platinum containing agent, can be selected to treat a complication of the cancer, or to provide relief to a subject from at least one symptom of the cancer, for example, sirolimus or rapamycin (Rapamune®), dexamethasone (Decadron®), palonosetron HCl (Aloxi®), aprepitant (Emend®), ondansetron (Zofran®), granisetron (Kytril®) or radiation.

Anti-cancer medicaments that can be orally administered are listed in Table 1, below.

TABLE 1
Orally Administrable Agents

altretamine
anagrelide
anastrozole
(ZD1033)
bexarotene
bicalutamide
capecitabine
clodronic acid
cytarabine
ocfosfate
dasatinib
dutasteride
erlotinib
exemestane
fadrozole
finasteride
fludarabine
gefitinib
GMDP
HMPL 002
hydroxycarbamide
ibandronic acid
idarubicin
imatinib
lapatinib
lenalidomide
letrozole
osaterone
polysaccharide K
prednimustine
S1
(gimeracil/oteracil/tegafur)
sobuzoxane
sorafenib
sunitinib
tamibarotene
tamoxifen
tegafur/uracil
temozolomide
thalidomide
topotecan
toremifene
treosulfan
trilostane
ubenimex
vinorelbine
vorinostat

Orally active anticancer agents include altretamine (Hexalen®), an alkylating agent; capecitabine (Xeloda®), an anti-metabolite; dasatinib (Sprycel®), a TK inhibitor; erlotinib (Tarceva®), an EGF receptor antagonist; gefitinib (Iress®), an EGF inhibitor; imatinib (Gleevec®), a TK inhibitor; lapatinib (Tykerb®), an EGFR inhibitor; lenalidomide, (Revlimid®), a TNF antagonist; sunitinib (Sutent®), a TK inhibitor; S-1 (gimeracil/oteracil/tegafur), an anti-metabolite; sorafenib (Nexavar®), an angiogenesis inhibitor; tegafur/uracil (UFT®), an anti-metabolite; temozolomide (Temodar®), an alkylating agent; thalidomide (Thalomid®), an angiogenesis inhibitor; topotecan (Hycamtin® for injection or Oral Hycamtin®), vinorelbine (Navelbine®), an anti-mitotic; cediranib (AZD2171, Recentin®), a VEGF inhibitor; and/or vorinostat (Zolinza®), a histone deacetylase inhibitor.

The term “tumor” herein refers to a malignant neoplasm of solid tissue.

As used herein, “refractory” refers to patients and their tumors wherein the tumor is unresponsive to first-line therapy, or to a patient or their tumor wherein the tumor recurs or progresses during the course of the first-line therapy.

A cancer that initially responds to therapy but then progresses after cessation of the therapy is referred to herein as “progressive.”

The term “controlled” includes complete response, partial response, or stable disease.

A “patient” as defined herein is a human being afflicted with cancer, such as a solid tumor, e.g., SCLC, NSCLC, colon cancer, prostate cancer, or the like.

The terms “first-line therapy” or “adjuvant therapy” refer to any non-platinum or organoplatinum-based chemotherapy, or radiotherapy, that is known in the art to be applicable for use, for example, chemotherapy using organoplatinum compounds such as cisplatin, carboplatin, satraplatin, or oxaliplatin, or other organoplatinum compounds. First-line therapy can also include administration of picoplatin. First-line therapy can also include administration of non-platinum anticancer agents such as etoposide, taxanes (paclitaxel/docetaxel; by the term “paclitaxel/docetaxel” is meant paclitaxel or docetaxel, or both), irinotecan, topotecan, doxorubicin such as pegylated liposomal doxorubicin, pemetrexed, vinorelbine, gemcitabine, 5-fluorouracil (5-FU), leucovorin, Erbitux® (cetuximab), Avastin® (bevacizumab) and the like.

The term “second-line therapy” refers to therapy administered to patients who have already received a course of treatment for the cancer, which can include radiation and/or therapy with non-platinum agents or with other organoplatinum agents such as cisplatin, carboplatin, oxaliplatin, satraplatin, and the like. Second line-therapy is medically indicated when the cancer is refractory or progressive after first-line therapy.

In various embodiments, methods of treatment are provided for various specific types of cancer using the inventive stabilized dosage form of picoplatin or a stabilized dosage form of picoplatin prepared by an inventive method. Optionally, a second anticancer drug can be administered in conjunction with the stabilized picoplatin dosage form. For example, pegylated liposomal doxorubicin can be administered in conjunction with the stabilized picoplatin dosage form. The stabilized picoplatin dosage form and the optional second anticancer agent each be administered parenterally, such as intravenously, or can be administered orally, in any combination.

The patient to whom the inventive stabilized picoplatin dosage form is administered can be chemotherapy-naïve (i.e., is receiving first-line therapy), or the patient can have previously received chemotherapy (i.e., is receiving second-line picoplatin therapy). For example, the patient's cancer can have already have developed resistance to organoplatinum anticancer agents other than picoplatin, such as cisplatin, carboplatin, oxaliplatin, satriplatin, and the like.

In various embodiments, picoplatin can be administered in low doses, for example the picoplatin can be administered at doses of 40-60 mg/m² of picoplatin every two weeks.

For example, as disclosed in U.S. Ser. No. 11/982,839, filed Nov. 5, 2007 by the inventors herein, picoplatin can be used in the treatment of small cell lung cancer (SCLC). The invention herein provides a method of treatment and a dosage form suitable for treatment of progressive small cell lung cancer (SCLC) or NSCLC. For example, if the first-line chemotherapy regimen includes administration of cisplatin, carboplatin, satraplatin, or oxaliplatin, and the SCLC is responsive to that treatment, but then progresses within, e.g., 180 days following cessation of the first-line treatment (i.e., is a progressive cancer), such a tumor can be treated with picoplatin as described herein.

In various embodiments, wherein the cancer comprises small cell lung cancer (SCLC), the method comprising:

(a) selecting a patient afflicted with small cell lung cancer; and

(b) administering to the patient the stabilized dosage form of picoplatin and optionally, etoposide, irinotecan, topotecan, paclitaxel, doxorubicin and/or amrubicin.

In various embodiments, wherein the cancer comprises non-small cell lung cancer (NSCLC), the method comprising:

(a) selecting a patient afflicted with non-small cell lung cancer; and

(b) administering to the patient picoplatin, and one or more of vinorelbine, pemetrexed, erlotinib, bevacizumab, gemcitabine, and paclitaxel/docetaxel.

The patient undergoing the treatment may also be suffering from forms of cancer or tumors in addition to the progressive SCLC; for example, the patient can also be suffering from a mixed tumor type comprising SCLC with non-small cell lung cancer (NSCLC), as well as having metastatic tumors.

The invention herein further includes a method of treating a progressive SCLC or other cancer wherein an effective anti-emetic amount of a 5-HT₃ receptor antagonist and dexamethasone are administered to the patient prior to administration of the picoplatin, or second agent(s), in order to reduce the side effects of nausea and vomiting that can accompany administration of anti-cancer compounds. An example of a 5-HT₃ receptor antagonist that can be used according to the invention is ondansetron.

In various embodiments, wherein the cancer comprises pancreatic cancer, the method comprising:

(a) selecting a patient afflicted with pancreatic cancer; and

(b) administering to the patient picoplatin, and one or more of gemcitabine, erlotinib, leucovorin, capecitabine, docetaxel and 5-FU.

In various embodiments, wherein the cancer comprises gastrointestinal cancer or gastric cancer, the method comprising:

(a) selecting a patient afflicted with gastrointestinal cancer; and

(b) administering to the patient picoplatin, and one or more of 5-FU, leucovorin, capecitabine, bevacizumab, cetuximab, irinotecan, epirubicin, imatinib, sunitinib and paclitaxel/docetaxel.

An embodiment of the present invention provides a method of treatment of hormone refractory prostate cancer, comprising administering to a human patient afflicted with hormone refractory prostate cancer, the cancer being metastatic and chemotherapy-naive, substantially concurrently, an inventive stabilized dosage form of picoplatin and docetaxel, with prednisone, wherein a dose of picoplatin of at least 120 mg/m² and a dose of docetaxel of about 60-100 mg/m² is administered intravenously at least once. The picoplatin and docetaxel can be administered at least twice, or can be administered about 2-12 times. Picoplatin, prednisone, and docetaxel can be administered at intervals of about 3-6 weeks.

In another embodiment of the invention, a method of treatment of hormone refractory prostate cancer, comprising administering to a human patient afflicted with hormone refractory prostate cancer, the cancer being metastatic and chemotherapy-naive, substantially concurrently, picoplatin and a taxane such as paclitaxel and/or docetaxel, wherein the docetaxel is administered at a dosage of about 60-100 mg/m² and the picoplatin is administered at a dosage of about 120-180 mg/m² is provided

One embodiment of the invention comprises the further administration of prednisone, the prednisone being administered to the patient orally at least once daily, e.g., twice daily. In one embodiment of the present method, the picoplatin and the docetaxel are both administered at intervals of about every three weeks, for example, 2 to 12 times (6 to 36 weeks), e.g., up to about ten times. The present method can extend the duration of life of the patient relative to the duration of life of a comparable patient not receiving the treatment, and can improve the quality of life of the patient relative to the quality of life of a comparable patient not receiving the treatment, and reduce the degree of pain felt by the patient relative to the degree of pain felt by a comparable patient not receiving the treatment. The present method can also reduce the level of prostate-specific antigen of the patient relative to the level of prostate-specific antigen of a comparable patient not receiving the treatment, and thus act to stabilize the disease.

The present dosage form is also useful in a method of treatment of hormone refractory prostate cancer, comprising:

(a) selecting a patient afflicted with metastatic hormone refractory prostate cancer; and

(b) administering to the patient picoplatin and docetaxel, and, optionally, bevacizumab.

The picoplatin and the docetaxel can exhibit additive or synergistic therapeutic effects on the patient. Little or no neurotoxicity is observed, and prostate-specific antigen (PSA) levels can be significantly reduced.

Preferably the picoplatin is administered concurrently (simultaneously or overlapping) or prior to the administration of the taxane. If the taxane is administered prior to the picoplatin, it is preferably administered about 10 hours to 5 minutes prior to the picoplatin, e.g., about 1 hour to 15 minutes prior to the picoplatin.

The invention herein provides a method of treatment and a dosage form suitable for treatment of ovarian cancer. For example, if the first-line chemotherapy regimen includes administration of cisplatin, carboplatin, satraplatin, or oxaliplatin, and the ovarian cancer is responsive to that treatment, but then progresses following cessation of the first-line treatment, such a tumor can be treated with picoplatin as described herein.

The present dosage form is also useful in a method of treatment of ovarian cancer, comprising:

(a) selecting a patient afflicted with ovarian cancer; and

(b) administering to the patient picoplatin, and, preferably, following the picoplatin, at least one of paclitaxel or docetaxel, and pegylated liposomal doxorubicin (“PLD” or “liposomal doxorubicin”).

If the first-line chemotherapy regimen includes administering of a platinum-containing anti-cancer agent such as cisplatin, carboplatin, satraplatin, or oxaliplatin and the ovarian cancer is resistant to that treatment or responds to that treatment but recurs during or within 90 days after cessation of treatment, it is said to be “refractory”.

If the first-line chemotherapy regimen, is responsive to that treatment but then progresses within 91-180 days (3-6 months) following cessation of the first-line treatment, it is said to be “progressive”.

If the first-line chemotherapy regimen, is responsive to that treatment but then progresses within in a period greater than 180 days (6 months) following cessation of the first-line treatment, it is said to be “recurrent”.

“CA-125” is an abbreviation for “cancer antigen 125” and is a mucinous glycoprotein and the product of the MUC16 gene. It is a tumor marker or biomarker that may be elevated in the blood of some people with specific types of cancers. CA-125 is clinically approved for following the response to treatment and predicting prognosis after treatment. It is especially useful for detecting the recurrence of ovarian cancer. While 79% of all ovarian cancers are positive for CA-125, the remainder do not express this antigen at all.

“Substantially concurrently” means in a simultaneous, sequential, or separate manner. As used herein, the substantially concurrent administering of picoplatin and liposomal doxorubicin hydrochloride means that each component is present in vivo at a therapeutically effective concentration at the same time. Thus, depending for example on the pharmaco-kinetics of the individual components and the administration route, the individual agents may be dosed sequentially, preferably separately (with a gap of, for example, 5 minutes to 1 hour), and this may effectively achieve an in vivo profile for the combination equivalent, or similar, to that achieved by simultaneous administration. A person skilled in monitoring the administering of the combination will readily be able to ascertain whether the components are present in vivo at the same time using standard techniques.

Doxorubicin hydrochloride is the established name for (8S,10S)-10-[(3-amino-2,3,6-trideoxy-a-L-lyxohexopyranosyl)oxy]-8-glycolyl-7,8,9,10-tetrahydro-6,8,11-trihydroxy-1-methoxy-5,12-naphthacenedione hydrochloride. It is an anthracycline topoisomerase inhibitor isolated from Streptomyces peucetius var caesius. The molecular formula of the drug is C₂₇H₂₉NO₁₁HCl; its molecular weight is 579.99.

Liposomal doxorubicin hydrochloride is distributed under the trade name DOXIL® and is distributed by Ortho Biotech Products LP (Raritan, N.J.). Each 10 mL vial contains 20 mg of doxorubicin hydrochloride at a concentration of 2 mg/ml (10 mL fill volume). Each 30 mg vial contains 50 mg of doxorubicin hydrochloride at a concentration of 2 mg/mL (25 mL fill volume).

Liposomes are microscopic vesicles composed of a phospholipid bilayer that are capable of encapsulating active drugs. The STEALTH® liposomes of DOXIL are formulated with surface-bound methoxypolyethylene glycol (MPEG), a process often referred to as pegylation, to protect liposomes from detection by the mononuclear phagocyte system (MPS) and to increase blood circulation time.

STEALTH® liposomes have a half-life of approximately 55 hours in humans. They are stable in blood, and direct measurement of liposomal doxorubicin shows that at least 90% of the drug (the assay used cannot quantify less than 5-10% free doxorubicin) remains liposome-encapsulated during circulation. It is hypothesized that because of their small size (ca. 100 nm) and persistence in the circulation, the pegylated DOXIL® liposomes are able to penetrate the altered and often compromised vasculature of tumors.

The dose of picoplatin, administered as a single dose, is generally from about 60 to 150 mg/m², and preferably at about 120 mg/m². The dose of liposomal doxorubicin hydrochloride, administered with the picoplatin as a single dose, is generally from about 20 to about 60 mg/m² of doxorubicin hydrochloride and preferably at about 40 mg/m² of doxorubicin hydrochloride. A preferred treatment is administration of picoplatin at a dosage of about 120 mg/m² and the liposomal doxorubicin hydrochloride at a dosage of about 40 mg/m² of doxorubicin hydrochloride.

These doses of picoplatin and liposomal doxorubicin hydrochloride can be administered to the patient at intervals of about once every 3 to about 6 weeks; each of such administrations constituting one treatment. Preferably, the treatments are about 4 weeks, (about 28 days) apart. The combination of picoplatin and liposomal doxorubicin hydrochloride can be administered at least twice, or can be administered for about 2 to about 10 treatments. Typically, the combination is administered for about 6 to about 7 treatments.

The picoplatin is administered to the patient shortly before, simultaneously with, or shortly after the administration of liposomal doxorubicin hydrochloride (i.e., substantially concurrently). The picoplatin may be administered in any manner that makes it systemically available for transport to the site of the cancer such as parenterally and orally. One preferred method is for the patient to receive picoplatin over 1 to 2 hours as an intravenous infusion followed by liposomal doxorubicin hydrochloride intravenously infused over 1 hour. The time between the end of the administration of the first drug and the start of the administration of the second drug should be no more than about 1 to about 3 hours, preferably between 5 minutes and 1 hour, (e.g. less than 1 hour).

It is believed that cancer patients suffering, refractory, progressive, or recurrent ovarian cancer can be treated more effectively with the combination of picoplatin and liposomal doxorubicin hydrochloride instead of either liposomal doxorubicin hydrochloride (e.g., DOXIL®) alone or the combination of liposomal doxorubicin hydrochloride and previously used platinum-containing anti-cancer agents, such as cisplatin, carboplatin, oxaliplatin, satraplatin, and lobaplatin, because they will experience fewer side effects, such as neuropathy, while preferably receiving higher doses of the platinum (Pt) drug. The administration of picoplatin in effective dosages, e.g., at about 75-120 mg/m², can reduce the incidence of side effects observed when liposomal doxorubicin (e.g., DOXIL) is administered simply, or with other anti-cancer drugs. Such side effects include hypersensitivity and Hand-Foot Syndrome, including desquamation, indicative of severe skin toxicity. This condition can be eliminated or substantially reduced by the picoplatin co-administration, so that the clinical regimen does not have to be interrupted or reduced.

It is further believed that at least an additive, and preferably a synergistic effect can be achieved with the substantially concurrent administration of picoplatin and liposomal doxorubicin hydrochloride.

In another embodiment of the present invention, picoplatin and liposomal doxorubicin hydrochloride are administered to the patient, as the only chemical anti-cancer agents, in conjunction with a regimen of best supportive care (BSC). Best supportive care for ovarian cancer comprises a number of palliative treatments that may also have therapeutic efficacy against ovarian cancer but are not considered curative. For example, in one embodiment of the invention, BSC includes one or more, and preferably all of irradiation to control symptoms of metastatic cancer, administration of analgesics to control pain, management of constipation, and treatment of dyspnea and treatment of anemia so as to maintain hemoglobin levels (≧90 g/L, i.e., ≧9 g/dL). The general guidelines used to provide subjects with best supportive care (BSC) are based on the NCCN Clinical Practice Guidelines for Ovarian Cancer (V.I.2008) <http://www.nccn.org/professionals/physician_gls/PDF/ovarian.pdf>and on the NCCN Clinical Practice Guidelines in Oncology—Palliative Care (V.I.2007) <http://www.nccn.org/professionals/physician_gls/PDF/palliative.pdf>.

It is believed that the substantially concurrent administration of picoplatin and liposomal doxorubicin hydrochloride will result in an increase in the duration of life of a patient is relative to the duration of life of a comparable patient not receiving the treatment. It is also believed that quality of life of a patient will be improved relative to the quality of life of a patient prior to the administration of the picoplatin and the liposomal doxorubicin hydrochloride. It is further believed that the degree of pain felt by a patient will be reduced relative to the degree of pain felt by a patient prior to the administration of the picoplatin and the liposomal doxorubicin hydrochloride. It is still further believed that the level of CA-125 cancer antigen of a patient will be decreased relative to the level of CA-125 cancer antigen of a comparable patient not receiving the treatment, and that the overall response (i.e., partial responses plus complete responses plus stable disease) will be increased.

The method of treating ovarian cancer can further comprise administering an anti-emetic therapy to the patient, either within about 30 minutes prior to or, substantially concurrently with, administration of the picoplatin and liposomal doxorubicin hydrochloride. The anti-emetic therapy can include administration of a corticosteroid or a 5-HT₃ receptor antagonist, or both. For example, the corticosteroid can be dexamethasone. The 5-HT₃ receptor antagonist can be palenosetron or ondansetron. Such compounds are effective in reducing the side effects of nausea and vomiting that can accompany administration of organoplatinum compounds. Additional anti-emetic agents can be administered, such a tranquilizer, for example, lorazepam.

The present invention further provides a kit comprising packaging containing, separately packaged, a sufficient number of unit dosage forms of picoplatin and unit dosage forms of liposomal doxorubicin hydrochloride to provide for a course of treatment for a human afflicted with ovarian cancer. A kit can further comprise instructional materials, such as instructions directing the dose or frequency of administration. For example, a kit can comprise sufficient doses of picoplatin and liposomal doxorubicin hydrochloride for one or more treatments. The unit dosage forms can be packaged separately, but in proximity, as in a blister pack.

The following examples are provided to illustrate the practice of the present invention and the invention is not meant to be limited thereby.

### Example 1

### Phase III Trial of Picoplatin and Liposomal Doxorubicin Hydrochloride to Treat Ovarian Cancer

This Phase III trial is designed to demonstrate that the combination of picoplatin and doxorubicin liposome hydrochloride both administered intravenously, results in improved progression free survival (PFS) compared to the use of liposomal doxorubicin hydrochloride used alone as a single anti-cancer agent in therapy for subjects with platinum resistant or refractory ovarian cancer. It is designed to compare the efficacy and safety of these two regimes as second-line therapy for subjects with ovarian or primary peritoneal carcinoma (OvCa).

Subjects with ovarian cancer that is resistant or refractory to initial chemotherapy will be enrolled in the study. Resistant or refractory is defined as the cancer having progressed within 6 months of completing first-line, platinum-containing chemotherapy will be enrolled in the study.

Approximately 350 subjects will be enrolled in this study. Subjects will be stratified by Eastern Cooperative Oncology Group Scale of Performance Status, (ECOG) performance status (PS) (0 vs. 1) and by whether or not they have radiologically measurable disease by RECIST (with or without CA-125 elevation) versus CA-125 elevation alone.

Subjects to be Included in the Study are those that Exhibit the Following:
Histological or cytological diagnosis of epithelial ovarian, fallopian tube or primary peritoneal carcinoma.
At least one, but no more than two prior chemotherapy regimens.
First-line chemotherapy that was platinum-based and intended to deliver cisplatin, at least 75 mg/m², at least every 4 weeks, or
   carboplatin, AUC at least=5, at least every 4 weeks, and included at least one additional drug, preferably a taxane
   included at least 2 treatments of first-line platinum based chemotherapy in the event of progressive disease, or
   included at least 3 treatments of first-line platinum based chemotherapy in the event of stable disease.
Radiological or CA-125 evidence of OvCa that never responded to first-line therapy (refractory); or responded initially to first-line therapy but progressed within 180 days of the final dose of platinum chemotherapy (resistant).
CT scans of pelvis and abdomen with contrast, preferably within 14 days prior to randomization (up to 21 days is allowed if necessary). MRI is acceptable in the case of allergy to contrast agents. The presence or absence of measurable disease by RECIST must be documented from the baseline CT or MRI scan.
In the absence of measurable disease by RECIST, the CA-125, measured on two occasions at least one week apart, must be
   greater than or equal to twice the upper limit of normal (ULN) in subjects whose CA-125 is below the upper limit of normal during prior therapy, or
   greater than or equal to twice the lowest value achieved with prior therapy in subjects whose CA-125 never normalized during prior therapy.
Eastern Cooperative Oncology Group Scale of Performance Status, (ECOG PS) 0 or 1 within 3 days prior to randomization.
At least 21 days must have elapsed since the most recent prior chemotherapy dose, with evidence of hematological recovery.
At least 14 days must have elapsed since the most recent prior radiotherapy dose.
At least 14 days must have elapsed since prior surgery except for the placement of venous access device.
Subject must be recovered to less than or equal to Grade 1 toxicity from all non-hematological adverse effects of prior therapies (excluding alopecia).
Age 18 years or over.
Average Neutrophil Count (ANC) greater than or equal to 1.5×10⁹/L (without growth factor support).
Platelet count greater than or equal to 100×10⁹/(without transfusion support).
Hemoglobin of greater than or equal to 90 g/L (transfusion or growth factors permitted to achieve this hemoglobin).
Aspartate aminotransferase, alanine aminotransferase, and lactate dehydrogenase levels less than or equal to 2.5 times the upper limit of normal or less than or equal to 5 times the upper limit of normal if liver involvement is present.
Bilirubin of less than or equal to 1.5 times the upper limit of normal.
Creatinine less than or equal to 1.5 times the upper limit of normal (hypovolemic subjects may be hydrated to achieve this level of blood urea nitrogen (BUN).
Women of childbearing potential must have a negative pregnancy test (serum or urine). Sexually active couples of child-bearing potential must agree to use appropriate birth control methods during chemotherapy and for 3 months after chemotherapy.
Signed informed consent.
   Subjects to be Excluded from the Study are those that Exhibit any of the Following:
Prior radiotherapy to the breast, skin, head or neck within the past 3 years or any previous radiation to mediastinum, abdomen or pelvis.
Tumor of low malignant potential (borderline tumors).
Prior treatment with liposomal doxorubicin.
Prior treatment with more than two different chemotherapy regimens.
Grade 2 or higher peripheral neuropathy.
Significant cardiac disease, defined as myocardial infarction within 3 months prior to randomization, congestive heart failure classified by the New York Heart Association as Class III or IV (Appendix V), uncontrolled cardiac arrhythmias, poorly controlled or unstable angina, or electrocardiographic evidence of acute ischemia.
Serious medical or psychiatric illness that could potentially interfere with the completion of study treatment according to this protocol, e.g., active infection, bowel obstruction, etc.
Use of other investigational drugs or tamoxifen within 30 days prior to randomization.
Subjects are not evaluable by CA-125 if they have received mouse antibodies (unless the assay used has been shown not to be influenced by human anti-mouse antibodies) or if there has been medical or surgical interference with their peritoneum or pleura during the previous 28 days.
Breast-feeding.
History of any other malignancy within 5 years, with the exception of treated non-melanoma skin cancer or carcinoma in situ of the cervix.

Subjects will receive computed tomography (CT) or magnetic resonance imaging (MRI) scans and CA-125 determinations will be performed for assessing the extent of the disease prior to the start of treatment. These will provide a baseline for evaluation during treatment.

Subjects may have measurable disease by RECIST criteria or assessable disease by CA-125 determination. In those with elevated CA-125 but no measureable disease by CT scan criteria, the CA-125 must be ≧100 U/mL (in those subjects whose CA-125 decreased to normal with initial chemotherapy) or have double from the lowest value achieved by chemotherapy.

After stratification, subjects will be centrally randomized 1:1 to receive either the combination of picoplatin intravenously and liposomal doxorubicin hydrochloride intravenously; or liposomal doxorubicin hydrochloride intravenously alone. Approximately 175 subjects will be assigned to each treatment. Subjects will be treated about every four weeks (about 28-days) until objective demonstration of disease progression. Both subject and treating investigator will remain blinded to treatment assignment until after documentation of progressive ovarian cancer.

Subjects randomized to receive the combination therapy will receive picoplatin, 120 mg/m² administered as a 1-2 hour intravenous infusion followed by liposomal doxorubicin hydrochloride, 40 mg/m² of doxorubicin hydrochloride, administered intravenously over 1 hour on Day 1 of a 28-day treatment cycle. Subjects randomized to receive only liposomal doxorubicin hydrochloride will receive a picoplatin placebo also administered as a 1-2 hour intravenous infusion followed by liposomal doxorubicin hydrochloride intravenously, containing 50 mg/m² of doxorubicin hydrochloride, administered over 1 hour on Day 1 of a 28-day treatment cycle.

All subjects will receive anti-emetic therapy consisting of a 5-HT₃ receptor antagonist plus dexamethasone immediately prior to chemotherapy. Anti-emetic therapy will be provided as needed thereafter.

Evaluations will include assessment of adverse events (AEs), and hematology values. White blood counts and platelet counts are also required between Day 11-15 of treatments 1 and 2 and during any treatment period for which dose reduction is required for hematological toxicity. CA-125 determination and CT scans or other assessments of tumor response will be performed every 8 weeks or after every other chemotherapy treatment until disease progression. Baseline and CA-125 determinations during the study will be performed by a central laboratory.

Subjects may continue to receive treatments of the combination of picoplatin and liposomal doxorubicin hydrochloride as long as they tolerate the therapy well and do not have progressive ovarian cancer. All clinical evidence of progression will be centrally reviewed by treatment-blinded independent reviewers.

### Criteria for Evaluation:

Efficacy: Efficacy will be assessed by analysis of the following endpoints.

Primary Endpoint: The primary efficacy endpoint will be Progression Free Survival (PFS).

Determination of disease progression will be made by independent, blinded, central review of radiographs using Response Evaluation Criteria in Solid Tumors (RECIST) and defined criteria for CA-125 progression: CA-125 value >100 U/mL and at least double the lowest value recorded on the study.

Secondary Endpoints:
The secondary endpoints will be the proportion of subjects who achieve a response as determined by RECIST criteria and CA-125 criteria, or any of:
(1) the proportion of subjects who achieve disease control (complete response plus partial response plus stable disease);
(2) the objective progression-free survival (RECIST criteria only); or
(3) overall survival (OS).

### Study Period and Survival Follow-Up Period:

All subjects will be considered “on-study” from the date of randomization until tumor progression, unacceptable toxicity, death, removal from study for other reasons or the end of the study.

It is estimated that the subjects for this study will be accumulated within 12 months. Allowing for a 6-month follow-up period after the last subject is randomized; the primary study endpoint should be determinable approximately 20 months after initiation. Follow-up for overall survival will be continued until 75% of subjects have died.

After discontinuation of study drugs or documentation of progressive disease, subjects will be followed for survival only.

### Safety:

The safety population will include all randomized subjects according to the treatment that each received in the study and will be used for all safety analysis.

Safety will be evaluated from the incidence of laboratory and non-laboratory adverse events, including serious adverse events. The severity of all adverse events will be evaluated according to the National Cancer Institute (NCI) Common Terminology Criteria for Adverse Events (CTCAE) Grading Scale, version 3. Adverse events will be recorded from the day of randomization until death or discontinuation from study or the end of the study. Serious adverse events that occur within 30 days of the last administration of study drug must be reported within 24 hours of identification to Poniard or its designee.

An embodiment of the present invention provides a method of treatment of colorectal cancer, comprising administering to a patient afflicted with metastatic colorectal cancer the stabilized dosage form of picoplatin, 5-fluorouracil (5-FU), and leucovorin (LV), wherein 5-FU and LV are administered intravenously and the picoplatin is administered with the LV and 5-FU every other time that the 5-FU and LV are administered. The picoplatin and the 5-FU/LV can exhibit additive or synergistic therapeutic effects on the patient. In one embodiment, the agents are administered at least twice at intervals, e.g., about 2-6 weeks.

Another embodiment of the invention provides a method of treatment of colorectal cancer, comprising administering to a patient afflicted with metastatic colorectal cancer effective amounts of a combination of the stabilized dosage form of picoplatin, 5-FU and leucovorin, wherein the picoplatin, 5-FU and leucovorin are administered intravenously at least twice at intervals of about two weeks, wherein the amount of picoplatin is less than the maximum tolerated dose of picoplatin when administered in said combination.

Another embodiment of the invention provides a method of treatment of colorectal cancer, comprising administering to a patient afflicted with metastatic colorectal cancer the stabilized dosage form of picoplatin, 5-FU, and leucovorin, wherein 5-FU and leucovorin are administered intravenously at intervals of about two weeks, and the picoplatin is administered with the leucovorin and 5-FU every time that the fluorouracil and leucovorin are administered, wherein the picoplatin is administered at a dose of about 45-180 mg/m², without dose-limiting toxicity It is unexpected that dosages would be as high as the upper limit when administration is biweekly.

In one embodiment of the present method, the patient preferably has not previously had systemic treatment, such as chemotherapy, for metastatic disease. The patient may have, however, received earlier adjuvant therapy at the time of primary tumor treatment, at least 6 months prior to the present picoplatin treatment.

In another embodiment of the invention, the picoplatin is administered substantially concurrently with the leucovorin and the picoplatin is administered at every second treatment of the patient with the 5-FU and the leucovorin, e.g., every four weeks. The leucovorin can be administered at a dosage of about 250-500 mg/m², preferably at about 400 mg/m². The picoplatin is administered at a dosage of about 60-180 mg/m². The 5-FU is administered at a total dosage of about 2500-3000 mg/m². A treatment cycle for leucovorin and 5-FU is every two weeks, and picoplatin is administered every 4 weeks, e.g., at a high dose of about 120-180 mg/m², preferably about 120-150 mg/m², e.g. about 150 mg/m².

Therefore, in one embodiment of the invention, the leucovorin, at a dosage of 250-500 mg/m², is administered as an about 2 hour infusion concurrently with the picoplatin, when it is given, wherein the picoplatin dosage is 120-180 mg/m², e.g., about 150 mg/m²; the administration of the leucovorin and the picoplatin being followed by a 5-FU dosage of about 400 mg/m² as a bolus; the 5-FU dosage being followed by 5-FU at a dosage of 2,400 mg/m², preferably administered as a 46 hour continuous infusion, wherein the leucovorin and 5-FU are provided to the patient at intervals of two weeks and the leucovorin, picoplatin, and 5-FU are provided to the patient at alternating intervals of four weeks.

In another embodiment of the invention, the leucovorin, at a dosage of 400 mg/m², is administered as a 2 hour infusion; the administration of the leucovorin being followed by a 5-FU bolus at a dosage of 400 mg/m²; the 5-FU bolus dosage being followed by parenteral 5-FU at a dosage of 2,400 mg/m², preferably administered as a 46 hour continuous infusion; the administration of the leucovorin and the 5-FU taking place every two weeks; wherein every two weeks picoplatin, is administered concurrently with the leucovorin, preferably simultaneously. Picoplatin dosages of about 45-180 mg/m² can be administered, without dose-limiting toxicity.

It has unexpectedly been found that, in some cases, the combination of low doses of picoplatin administered with leucovorin and 5-FU at every treatment cycle, are as effective as, or more effective than, higher doses, e.g., the MTD, given at the same intervals, in producing a response. The MTD for the 2 week and 4 week picoplatin administration schedules (see Table 1) are discussed below. Preferably, such doses in the initial treatment are lower or substantially lower than the MTD. Such doses can range from about 40-60 mg/m² of picoplatin every two weeks, given with leucovorin and followed by 5-FU, as discussed below.

The present dosage form is also useful in a method of treatment of colorectal cancer, comprising:

(a) selecting a patient afflicted with metastatic colorectal cancer; and

(b) administering to the patient picoplatin, and one or more of 5-fluorouracil, and leucovorin, and optionally, at least one of bevacizumab, cetuximab, panitumumab, radiation, and capecitabin. In one embodiment, the picoplatin and the second agent(s) are administered at least twice, e.g., at about 2-6 week intervals.

For example, the leucovorin, at a dosage of about 400 mg/m², is administered as a 2 hour infusion concurrently with the picoplatin, each from a separate container, wherein the picoplatin dosage is about 45-180 mg/m²; the administration of the leucovorin and the picoplatin being followed by a 5-fluorouracil bolus at a dosage of about 400 mg/m²; the 5-fluorouracil bolus being followed by 5-fluorouracil at a dosage of about 2,400 mg/m² administered as a 46 hour continuous infusion; wherein the leucovorin, picoplatin, and 5-fluorouracil are provided to the patient every two weeks. Alternatively, the picoplatin may be administered with the other agents every 4 weeks.

Picoplatin and/or the second agents are preferably administered at least twice at effective intervals, e.g., of 2-6 weeks. Picoplatin may be given concurrently with the second agent(s) or they may be alternated, or picoplatin may be alternated with picoplatin and a second agent during the treatment cycles.

In various embodiments of the inventive methods of treatment, little or no neurotoxicity (i.e., no neurotoxicity of grade 3 or above), is observed to occur in the patient.

The efficacy of platinum analogues is limited by several (intrinsic or acquired) mechanisms of resistance, including impaired cellular uptake, intracellular inactivation by thiols (e.g., reduced glutathione) and enhanced DNA repair and/or increased tolerance to platinum-DNA adducts.

In various embodiments, the second anticancer agent can be gemcitabine, pegylated liposomal doxorubicin, vinorelbine, paclitaxel, topotecan, docetaxel, doxetaxel/prednisone, 5-fluorouracil/leucovorin, capecitabine, etoposide, bevacizumab, cetuximab, panitumumab, pemetrexed, amrubicin, or a combination thereof.

In various embodiments, the second anticancer agent can be camptothecin, capecitabine, irinotecan, etoposide, vinblastine, vindesine, cyclophosphamide, ifosfamide, or methotrexate, or a combination thereof.

The picoplatin, when administered parenterally in accord with the present invention is in an aqueous solution, preferably sterile. The aqueous solution can include a source of chloride ion, for example NaCl, such that the aqueous solution is stabilized against degradation. This concentration was unexpectedly found to stabilize the dissolved picoplatin, as discussed above. The aqueous solution is preferably free of preservatives such as chlorite or quaternary ammonium compounds due to the possibility of such preservatives reacting chemically with the picoplatin. The present solutions preferably do not include added preservatives, since they are inherently biocidal.

The picoplatin can be administered in doses ranging from about 60 mg/m² up to about 150 mg/m² per dose, or greater than 150 mg/m² per dose, for example, up to about 180 mg/m² per dose. These dosage units refer to the quantity in milligrams per square meter of body surface area. The starting dose will be based on the body surface area (BSA) which can be calculated from the height and weight of the subject at baseline according to the following equation:

$$BSA ( {m}^{2} ) = \sqrt{\frac{HEIGHT ( cm ) \times Weight ( kg )}{3600}}$$

Subsequent treatment cycles can use the BSA calculated for the starting dose. If the subject's weight changes by at more than 10%, the treating physician must recalculate the BSA and adjust the dose accordingly.

When the picoplatin is administered intravenously as an aqueous solution, for example at a concentration of 0.5 mg/mL in sterile isotonic water, it can be given over the period of about an hour or about two hours. The total amount of picoplatin per dose given to a patient can amount to about 200 to about 300 mg, for example, if given at a concentration of about 0.5 mg/mL in sterile isotonic water solution, the total dose can amount to about 400-600 mL of the solution, e.g., the contents of 2-3 IV dosage forms are administered.

The total number of doses of picoplatin that can be administered over a period of times can be in the range of two to about 14 separate doses, for example, about 5-7 doses, and the doses can be given at points in time about three weeks apart ranging up to about six weeks apart. However, the doses can be continued beyond up to a period of about a year provided that toxicity contraindicating the treatment does not appear.

The invention also provides a dosage form for picoplatin comprising, in a container, a solution in water, a chloride salt, and picoplatin at a concentration in the water of about 0.25-0.75 mg/ml (0.025-0.075 wt-%). This dosage form is suitable for the parenteral administration of effective dosages of picoplatin, each individual container containing about 100-125 mg of picoplatin, and being suitable for intravenous administration, e.g., for aseptic connection to IV valves, tubing, parts, lines and the like, or for transfer between infusion devices.

The container of the dosage form can be a glass infusion vial, a infusion bag formed of drug-resistant polymer, or a syringe formed of drug-resistant polymer, such as polymers that do not comprise halides, amines, or amides. As picoplatin is light-sensitive and can decompose when exposed to visible light, the container can be further contained in a secondary covering that is sufficiently opaque to reduce the incident light to an acceptable level.

If capped, the portions of the cap that contact the solution will not contain a redox active metal, such as may react with the picoplatin.

The chloride ion source can be any suitable Group I or II metal chloride; sodium chloride can be used, or alternatively potassium chloride, magnesium chloride, calcium chloride, or other biocompatible substances. The solution can be adjusted such that it is isotonic with human body fluids, e.g., with blood, spinal fluid, lymphatic fluid, and the like. Preferably, no preservative that could interact with the picoplatin component is included; chlorine, chlorite and quarternary ammonium salts (“quats”) should generally be avoided. The solution should be sterile, which may be accomplished by any of the various methods well known in the art such as ultrafiltration. Sterility within the container can be maintained through use of sterilized containers, with suitable closures such as ETFE copolymer-coated chlorinated butyl rubber stoppers and flip-off crimp seals. The solutions can be deoxygenated as needed.

The container of the dosage form can include a closure means such as a cap that provides identifying information useful to a care provider, such as a physician or a nurse, that can include the identity, concentration, expiration date. This can serve to avoid medical mistakes and to provide an additional level of assurance to the care provider and to the patient that the correct medication is being administered. The identifying information can be in a non-visual form so that it can be detected in low light, for example, by textural features of the cap, raised letters signifying picoplatin and the dosage, and the like. Alternatively, the cap can be colored in a manner that conveys dosing information or to identify the contents. For example, if a treatment session will use three containers, the containers can be coded, such as with different colors, to indicate to the care provider the relative position of a given container in the treatment sequence, first, second or third. This serves to avoid medical mistakes such as over- or under-dosing as could occur if the care provider loses count of the containers administered to a patient in a treatment session.

As a light-sensitive compound, picoplatin and its solutions are protected from light exposure, for example, by packaging in opaque materials. Thus, dosage forms of the present invention such as solutions held in containers, such as nominal 200 mL vials made of glass or of a polymer such as ethylene-vinyl acetate copolymer or polypropylene can be shielded from light by secondary packaging that minimizes exposure to visible light. Preferably, the package can be shaped so as to remain in place as a light-blocker while the solution is administered to the patient. Additionally, the container can be formed from light-protective material, such as amber glass.

Due to the light-sensitivity of the picoplatin, during preparation of the solution and filling of the containers, the process can be carried out under red-filtered light, for example, a photographic safe light, in order to avoid photolytic decomposition of the picoplatin.

The invention provides one or more of dosage forms packaged with instruction materials regarding administration of the dosage form., or with instruction materials that comprise labeling means, e.g., labels, tags, CDs, DVDs, cassette tapes and the like, describing a use of the dosage form that has been approved by a government regulatory agency.

Thus, the dosage form of the invention provides one or more unit dosage forms adapted to practice the method of the invention, incorporating the picoplatin at a suitable concentration in a biocompatible carrier that is packaged to maintain sterility and to protect the active ingredient against deterioration.

The invention further provides a kit adapted for a single course of treatment comprising two or more of the dosage forms further contained in packaging material. For example, the kit can include three dosage form units, each dosage form unit providing 200 ml of a solution comprising 100 mg of picoplatin, for a total of 300 mg picoplatin per kit, which suffices for at least one administration of a dose of picoplatin of up to 300 mg. The packaging material of the kit can be light-protective in order to avoid photolytic decomposition of the picoplatin. The kit can include packaging material such as shaped polystyrene foam that serves to protect the containers from damage, light, and thermal extremes. The kit can further include instruction means and labeling means, as well as accessories for administration of the container contents such as tubing, valves, or needles for IV administration.

The dosage form of the invention can further be packaged in multiple dosage forms adapted to practice the method of the invention. For example, two or three single-unit dosage forms can be packaged together as a “six-pack,” for example for shipment from a supplier to a medical facility providing treatment to patients, in a single container.

The kit can include separately packaged and labeled multiple or single use containers of non-platinum anticancer drugs and/or adjuvant agents intended to be administered parenterally before, concurrently with, or after the picoplatin, including potentiators, rescue agents or anti-emetics.

Useful agents for administration with picoplatin, methods of treatment, dosing regimens, and compositions are also disclosed in U.S. patent application Ser. Nos. 10/276,503, filed Sep. 4, 2003; 11/982,841, filed Nov. 5, 2007; 11/982,840, filed Nov. 5, 2007; 11/935,979, filed Nov. 6, 2007; 11/982,839, filed Nov. 5, 2007; 12/367,394, filed Feb. 6, 2009; 12/464,662, filed May 12, 2009; 12/465,563, filed May 13, 2009; 12/508,392, filed Jul. 23, 2009; 12/536,311, filed Aug. 5, 2009; 12/536,335, filed Aug. 8, 2009; in U.S. Pat. Nos. 7,060,808 and 4,673,668; in PCT WO/98/45331 and WO/96/40210 and in U.S. provisional application Ser. Nos. 60/889,171, filed Feb. 9, 2007; 60/889,681, filed Feb. 13, 2007; 60/857,067, filed Nov. 6, 2006; 60/877,515, filed Dec. 28, 2006; 60/927,347, filed May 5, 2007; 60/931,309, filed May 22, 2007; 60/969,441, filed Aug. 31, 2007; 60/857,017, filed Nov. 6, 2006; 60/857,564, filed Nov. 8, 2006; 60/877,570, filed Dec. 28, 2006; 60/889,179, filed Feb. 9, 2007; 60/890,950, filed Feb. 21, 2007; 60/931,609, filed May 24, 2007; 60/952,440, filed Jul. 27, 2007; 60/857,066, filed Nov. 6, 2006; 60/857,725, filed Nov. 8, 2006; 60/877,495, filed Dec. 28, 2006; 60/889,191, filed Feb. 9, 2007; 60/931,589, filed May 24, 2007; 60/983,852, filed Oct. 30, 2007; 60/889,201, filed Feb. 9, 2007; 60/889,675, filed Feb. 13, 2007; 60/984,156, filed Oct. 31, 2007; 60/989,020, filed Nov. 19, 2007; and PCT Pat. Ser. No. PCT/US2008/001746, filed Feb. 8, 2008, entitled “Encapsulated Picoplatin”, PCT Pat. Ser. No. PCT/US2008/001752, filed Feb. 8, 2008, entitled “Stabilized Picoplatin Oral Dosage Form,” PCT Pat. Ser. No. PCT/US2008/008669, filed Jul. 16, 2008, entitled “Oral Formulations for Picoplatin,” PCT Pat. Ser. No. PCT/US2009/000770, filed Feb. 6, 2009, entitled “Use of Picoplatin and Bevacizumab to Treat Colorectal Cancer,” PCT Pat. Ser. No. PCT/US2009/000773, filed Feb. 6, 2009, entitled “Use of Picoplatin and Cetuximab to Treat Colorectal Cancer,” PCT Pat. Ser. No. PCT/US2009/000750, filed Feb. 6, 2009, entitled “Picoplatin and Amrubicin to Treat Lung Cancer,” U.S. Ser. No. 60/950,033 filed Jul. 16, 2007 and U.S. Ser. No 61/043,962 filed Apr. 10, 2008, both entitled “Oral Formulations for Picoplatin”; U.S. Ser. No. 61/036,302, filed Mar. 13, 2008, entitled “Method of Treatment of Organoplatinum-Resistant Cancers”; and in Martell et al., U.S. provisional application Serial No. 61/027,387, filed Feb. 8, 2008, entitled “Use of Picoplatin and Bevacizumab to Treat Colorectal Cancer” (Atty. Docket No. 295.114PRV); Martell et al., U.S. provisional application Ser. No. 61/027,382, filed Feb. 8, 2008, entitled “Use of Picoplatin and Cetuximab to Treat Colorectal Cancer” (Atty. Docket No. 295.115PRV); Karlin et al., U.S. provisional application Ser. No. 61/027,360, filed Feb. 8, 2008, entitled “Picoplatin and Amrubicin to Treat Lung Cancer” (Atty. Docket No. 295.116PRV); U.S. provisional application Ser. No. 61/034,410, filed Mar. 6, 2008, entitled “Use of Picoplatin and Liposomal Doxorubicin Hydrochloride to Treat Ovarian Cancer” (Atty. Docket No. 295.117PRV); Martell et al., U.S. provisional application Ser. No. 61/027,388, filed Feb. 8, 2008, entitled “Combination Chemotherapy Comprising Stabilized Intravenous Picoplatin” (Atty. Docket No. 295.120PRV); Leigh et al., U.S. provisional application Ser. No. 61/186,526, filed Jun. 12, 2009, entitled “Improved Synthesis of Picoplatin” (Atty. Docket No. 295.132PRV); Phillips et al., U.S. provisional application Ser. No. 61/169,679, filed Apr. 15, 2009, and Ser. No. 61/170,487, filed Apr. 17, 2009, both entitled “Picoplatin Oral Dosage Form Having High Bioavailability” (Atty. Docket Nos. 295.133PRV and 295.133PV2); Karlin et al., U.S. provisional application Ser. No. 61/177,567, filed May 12, 2009, entitled “Use of Picoplatin to Treat Prostate Cancer” (Atty. Docket No. 295.136PRV); U.S. provisional application Ser. No. 61/177,571, filed May 12, 2009, entitled “Use of Picoplatin and Docetaxel to Treat Prostate Cancer” (Atty. Docket No. 295.137PRV); Leigh et al., U.S. provisional application Ser. No. 61/243,314, filed Sep. 17, 2009, entitled “Methods of Preparation of Organoplatinum-II Compounds” (Atty. Docket No. 295.141PRV): and Martell et al. U.S. provisional application Ser. No. 61/228,471, filed Jul. 24, 2009, entitled “Use of Picoplatin and Liposomal Doxorubicin Hydrochloride to Treat Ovarian Cancer” (Atty. Docket No. 295.144PRV).

All publications, patents, and patent applications are incorporated herein by reference. While in the foregoing specification of this invention has been described in relation to certain preferred embodiments thereof, and many details have been set forth for purposes of illustration, it will be apparent to those skilled in the art that the invention is susceptible to additional embodiments and that certain of the details described herein may be varied and modified considerably without departing from the basic principles, spirit, and scope of the invention.

What is claimed is:

1. A method of treating ovarian cancer, comprising:
administering to a human patient afflicted with ovarian cancer, substantially concurrently, picoplatin and pegylated liposomal doxorubicin hydrochloride, wherein the picoplatin is administered at least once at a dosage of at least about 60 mg/m² and the liposomal doxorubicin hydrochloride is administered at least once at a dosage of at least about 20 mg/m² of doxorubicin hydrochloride, up to the maximum tolerated dose of each agent in combination.

2. The method of claim 1 wherein the dosage of picoplatin is administered at between about 60 and about 150 mg/m² and the dosage of liposomal doxorubicin hydrochloride is administered at between about 20 and about 60 mg/m² of doxorubicin hydrochloride.

3. The method of claim 2 wherein the picoplatin is administered at a dosage of about 120 mg/m², either simultaneously with or followed up to no more than about 1 to 3 hours later by administration of liposomal doxorubicin hydrochloride intravenously infused in a dosage of about 40 mg/m² of doxorubicin hydrochloride over a period of about 1 hour.

4. The method of claim 3 wherein the picoplatin and the liposomal doxorubicin hydrochloride are both administered intravenously.

5. The method of claim 3 wherein the picoplatin is administered at a dosage of about 120 mg/m², either simultaneously with or followed up to no more than about 3 hours later by administration of liposomal doxorubicin hydrochloride at a dosage of about 40 mg/m² of doxorubicin hydrochloride infused over a period of about 1 hour.

6. The method of claim 1 wherein the picoplatin is administered prior to administration of the doxorubicin hydrochloride.

7. The method of claim 1 or 6 wherein the picoplatin and the liposomal doxorubicin hydrochloride are both administered about once every 3 to about 6 weeks.

8. The method of claim 7 wherein the picoplatin and the liposomal doxorubicin hydrochloride are both administered about every 4 weeks.

9. The method of claim 7 wherein the picoplatin and the liposomal doxorubicin hydrochloride are each administered at least twice.

10. The method of claim 7 wherein the picoplatin and the liposomal doxorubicin hydrochloride are each administered for about 2 to about 10 treatments.

11. The method of claim 1 or 6 wherein the picoplatin is administered at doses of about 110-120 mg/m².

12. The method of claim 1 or 6 wherein the level of CA-125 cancer antigen of the patient is decreased relative to the level of CA-125 cancer antigen of a comparable patient not receiving the treatment.

13. The method of claim 1 or 6 wherein an effective anti-emetic amount of a 5-HT₃ receptor antagonist and dexamethasone are administered to the patient prior to administration of the picoplatin or liposomal doxorubicin hydrochloride.

14. The method of claim 1 or 6 wherein the dosage of picoplatin reduces or eliminates at least one of the side effects associated with the doxorubicin hydrochloride.

15. The method of claim 14 wherein the side effect is Hand-Foot syndrome.
